(19) United States
(12) Patent Application Publication
(10) Pub. No.: US 2025/0303010 A1
(43) Pub. Date: Oct. 2, 2025

(54) SMART DRAIN COVER FOR SINK STERILIZATION AND MITIGATION OF AEROSOLIZED PATHOGENS

(71) Applicant: Oregon Health & Science University, Portland, OR (US)

(72) Inventors: Whitney MENZEL, Portland, OR (US); Albert CHI, Portland, OR (US); Justin FINK, Portland, OR (US); Lynne STRASFELD, Portland, OR (US)

(73) Assignee: Oregon Health & Science University, Portland, OR (US)

(21) Appl. No.: 19/089,404

(22) Filed: Mar. 25, 2025

Related U.S. Application Data
(60) Provisional application No. 63/570,983, filed on Mar. 28, 2024.

(51) Int. Cl. A61L 2/10; A61L 2/24; A61L 2/26; E03C 1/126

(52) U.S. Cl. CPC A61L 2/10; A61L 2/24; A61L 2/26; E03C 1/126

(57) ABSTRACT

Provided are drain cover devices that include a light emitting diode. The light emitting diode exhibits an average radiant light intensity, a wavelength, or both, configured for killing, destroying, rendering inert, rendering non-infectious, or a combination thereof, a pathogen, a fungi, a toxin, or a combination thereof. Drain cover devices include a housing having a top exhibiting a first diameter, a top surface, and an underside, bottom component, and a drain adapter. The bottom component is affixed to the underside of the top component and includes a bottom surface defining a bottom component central aperture and a second diameter. The drain adapter is affixed to the bottom component and defines a drain adapter and a central aperture, and the top and bottom component define a central volume. An electronic circuit board having the light emitting diode is disposed within the central volume.

### CROSS REFERENCE TO RELATED APPLICATIONS

This application claims priority to U.S. Provisional Application No. 63/570,983, filed Mar. 28, 2024, the disclosure of which is hereby incorporated by reference in its entirety for all purposes.

### FIELD

The present invention relates to devices, systems, and techniques for mitigating generation of aerosolized pathogens. This invention generally relates to covers for use in sterilization sink drains.

### BACKGROUND

For a number of years, researchers have been investigating potential sources of hospital-acquired infection-causing microorganisms such as carbapenem-resistant Enterobacteriaceae (CRE). CRE infections are capable of transferring their resistance genes from one bacterial species to another—for example, from carbapenem-resistant Klebsiella pneumoniae to Escherichia coli—resulting in difficult to treat conditions. As research expanded, sink drains in hospital rooms presented as a likely source of hospital-acquired infections. Through a process of elimination, it was determined that each sink in hospital patients' room was a possible source of infections. In some cases, bacteria can spread along common pipes connecting sinks and colonize the p-trap. Once there, they can form a biofilm which can grow upwards to reach the sink strainer at a rate of up to an inch per day if fed with nutrients such as foods and drinks from patients or family vising a patient.

Current methods for combating such events have included replacing the entire contaminated sink unit or replacing the downpipes and p-traps, pouring large amounts of disinfectants down the sink, and using devices that heat the sink and subjects the downpipe to ultrasound to kill and remove the biofilm. The methods currently used to combat such conditions are costly, environmentally unfriendly, or laborious and unsafe.

### SUMMARY

The present technology is generally directed to aspects of drain cover devices that include a light emitting diode, an electronic circuit board, and a housing. The light emitting diode includes an average radiant light intensity, a wavelength, or both an average radiant light intensity and a wavelength configured for killing, destroying, rendering inert, rending non-infection, or a combination thereof, a pathogen, a fungi, a toxin, or a combination thereof. The electronic circuit board includes one or more surfaces having light emitting diodes formed on the surface. The housing includes a top component, a bottom component, and a drain adapter. The top component includes a first diameter, a top surface, and an underside. The bottom component is affixed to the underside of the top component and includes a bottom surface defining a bottom component, a central aperture, and a second diameter. The first diameter is greater than the second diameter. The drain adapter is affixed to the bottom component and defines the drain adapter central aperture. The top component and the bottom component define a central volume that includes the electronic circuit board and the light emitting diode.

In embodiments, the light emitting diode includes an ultraviolet light having a wavelength of from 180 nm to 410 nm. Furthermore, in embodiments, the average radiant intensity of the light emitting diode is from about 0.100 mW/cm² to about 0.500 mW/cm². In more embodiments, the device includes a plurality of light emitting diodes in communication with the electronic circuit and positioned within the central volume. Additionally or alternatively, in embodiments, the electronic circuit board includes a top surface and a bottom surface, where the light emitting diode is formed on the bottom surface of the electronic circuit board. In embodiments, the bottom surface is disposed facing the bottom component of the central aperture. In yet more embodiments, the device includes an energy storage device and a charging circuit. In embodiments, the charging circuit includes an induction coil or a wired charging port. In further embodiments, the electronic circuit includes at least one of an indicator for communicating information about the device or electronic circuit, an inertial sensor, an orientation sensor, a position sensor, or a wireless transceiver. Embodiments include where the indicator is an audible indicator, a visual indicator, or a wireless signal including the formation about the device or electronic circuit. In embodiments, the information corresponds to a battery charge state, an orientation of the device, or an operation error of the device.

In embodiments, the device further includes where the top surface of the top component faces away from the bottom component, and the underside including a mounting bracket disposed along the underside at a diameter less than the first diameter. In embodiments, the diameter at which the mounting bracket is located is within about 5% of the second diameter. In further embodiments, the top surface of the top component includes a convex shape. Moreover, in embodiments, the drain adapter may include a diameter that is within about 5% of the second diameter.

In embodiments, the electronic circuit further includes a processor and a non-transitory computer-readable storage medium in data communication with the processor, where the non-transitory computer-readable storage medium stores processor executable instructions that, when executed by the processor, cause the processor to perform operations. The operations may include determining at least one property of the device and controlling a function of the device based on the at least one property. In embodiments, the at least one property is an orientation of the device, a battery life, an indication of a sterilization procedure, a status of a light output from the light emitting diode, a proximity or connection toa charging device or circuit. In more embodiments, the function of the devices activating emission from the light emitting diode include stopping emission from the light emitting diode, disabling or blocking emission from the light emitting diode, activating charging of an energy storage device, stopping charging or an energy storage device, or activating an audible alarm or indicator. In embodiments, the operations further include receiving instructions from a mobile application. The instructions include a parameter associated with a sterilization protocol. In embodiments, the parameter includes one of a length of time for sterilization, a frequency of sterilization, or a combination of a length of time and a frequency.

The present technology is also generally directed to methods of sterilizing a sink drain. Methods include positioning a device according to one or more of the above aspects over a sink drain such that the bottom surface central aperture, the drain adapter central aperture, or a combination thereof are disposed within or above the surface of a sink, or a surface of the sink drain, and initiating a sterilization protocol of the device. In embodiments, the sterilization protocol may include one or more of a time duration associated with emission of light from the light emitting diode, or a repetition frequency associated with emission of light from the light emitting diode. The sterilization protocol may correspond to a single duration of light emission from the light emitting diode per day sufficient to kill, destroy, and/or render inert or non-infectious the pathogen and/or the toxin, a periodically repeating duration of light emission from the light emitting diode per day sufficient to periodically or cumulatively kill, destroy, and/or render inert or non-infectious the pathogen and/or the toxin, or an on-demand duration of light emission from the light emitting diode per day for a duration of time sufficient to kill, destroy, and/or render inert or non-infectious the pathogen and/or the toxin. In embodiments, the method includes receiving a change to the sterilization protocol via a wireless or wired communication signal.

In embodiments, the method includes activating an indicator for communicating information about the device or electric circuit. In embodiments, the indicator includes an audible indicator, a visual indicator, or a wireless signal including the information about the device or electronic circuit. The information corresponds to a battery charge state, an orientation of the device, or an operation error of the device.

The present technology is also generally directed to a kit. The kit may include a sterilization device, where the sterilization device includes a light emitting diode. The light emitting diode includes a light intensity, wavelength, or both an intensity and a wavelength configured for killing, destroying, rendering inert, rendering non-infectious, or a combination thereof, a pathogen, a fungi, a toxin, or a combination thereof. The device further includes an electronic circuit board, wherein the light emitting diode is formed on one or more surfaces of the electronic circuit board, a housing, and a set of instructions for assembling and/or using the sterilization device. The housing includes a top component, a bottom component, and a drain adapter. The top component includes a first diameter, a top surface, and an underside. The bottom component is affixed to the underside of the top component and includes a bottom surface defining a bottom component, a central aperture, and a second diameter. The first diameter is greater than the second diameter. The drain adapter is affixed to the bottom component and defines the drain adapter central aperture. The top component and the bottom component define a central volume that includes the electronic circuit board and the light emitting diode.

In embodiments, the kit further includes a charging device configured to perform wired or wireless charging of a battery of the sterilization device. In further embodiments, the set of instructions includes one or more instructions for performing wired or wireless charging of the battery of the sterilization device using the charging device. Moreover, in embodiments, the kit further includes a UV test card and the instructions may include procedures for testing the sterilization device using the UV test card.

In embodiments, kits include as least a second bottom component or a second drain adapter. The second bottom component and/or the second drain adapter include a second leg arrangement or second support arrangement. The instructions include one or more instructions for replacing the bottom component with the second bottom component and/or replacing the drain component with the second drain component. In more embodiments, the instructions may describe operations of an audible low battery alarm of the sterilization device, an operation on an inertial sensor or orientation sensor of the sterilization device, a charging procedure for the battery of the sterilization device, a default sterilization protocol of the sterilization device, one or more alternative sterilization protocols of the sterilization device, instructions for changing sterilization protocols of the sterilization device, or a combination thereof. In embodiments, the kit includes a sterilization device according to any one or more of the above discussed aspects, and/or the device performs or is configured to perform any one or more aspects of methods discussed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a general schematic of a sink and drain system, according to one or more embodiments of the present disclosure.

FIG. 2 provides an illustration of an example diagram for a sink drain cover, according to one or more embodiments of the present disclosure.

FIG. 3A provides an illustration of an exemplary sink drain cover configured to fit within a recessed drain of a sink, according to one or more embodiments of the present disclosure.

FIG. 3B provides an illustration of an exemplary sink drain cover configured to fit over a non-recessed drain of a sink, according to one or more embodiments of the present disclosure.

FIG. 4A provides an illustration of an exemplary sink drain cover configured to fit within a recessed drain of a sink, according to one or more embodiments of the present disclosure.

FIG. 4B provides an illustration of an exemplary sink drain cover configured to fit over a non-recessed drain of a sink, according to one or more embodiments of the present disclosure.

FIG. 5A provides an illustration of an exemplary configuration of LEDs positioned on the bottom components of the sink drain cover, according to one or more embodiments of the present disclosure.

FIG. 5B provides an illustration of exemplary light emitted from the LEDs positioned along the bottom component of the sink drain, according to one or more embodiments of the present disclosure.

FIG. 6 provides various illustrations of exemplary LED light emission cones from the LED configured on the bottom component of the sink drain cover, according to one or more embodiments of the present disclosure.

FIG. 7 provide an illustration of an example array sterilization sequence of exemplary LEDs, according to one or more embodiments of the present disclosure.

FIG. 8 provides a general schematic of a block diagram of an example of a computing device usable for implementing one or more embodiments of the present disclosure.

FIG. 9 provides a general schematic of a block diagram of an example network usable for implementing one or more embodiments of the present disclosure.

### DETAILED DESCRIPTION

The present disclosure provides devices, kits, and methods for combating pathogen and toxin growth or buildup in the drain of a sink and attached downpipes leading to the trap. The devices and techniques described herein are designed for directing light down the sink drain such that the light may kill or destroy the pathogens and/or toxins and aid in preventing hospital-acquired infections. The sink drain cover, which may be configured from cost effective material, may include a housing that may be configurable to a drain located in the basin of a sink. The housing may have multicomponent housing, such as including a first component that is configured to physically connect to a second component. The second component may be configured to house the electronic circuit and may be in physical contact with the sink drain. The electronic circuit can be adapted for insertion into the housing and may be electrically coupled to at least one LED. The LED may be used to direct light down the sink drain that may be capable of killing, destroying, or rendering inert various pathogens and toxins. The sink drain cover device described herein may be a cost-effective and environmentally friendly method of preventing hospital-acquired infections or prevent pathogen and toxin growth or buildup in commercial and personal sinks.

Various features related to the methods and devices for sanitizing or disinfecting a sink and sink drain components are further explained in detail below.

### Terminology

Unless otherwise defined below, all technical and scientific terms used herein are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one skilled in the art. While the following terms are believed to be well understood, the following definitions are set forth to facilitate an explanation of the presently disclosed subject.

As used herein, the singular forms “a,” “an,” and “the” include plural referents unless the content dictates otherwise. Thus, for example, reference to “an antibody” can refer to any or all antibodies.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting of those certain elements.” As used herein, “and/or” refers to and encompasses any and all possible combinations of one or more of the associated listed items.

The terms “about” and “approximately” as used herein shall generally mean an acceptable degree of error for the quantity measured, given the nature or precision of the measurements. Exemplary degrees of error are within 20% (%), preferably within 10%, and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends.

The term “subject” means any animal, including any vertebrate or mammal, and, in particular, a human, and can also be referred to, e.g., as an individual or patient.

The term “biofilm” as used herein shall generally mean a community of microorganisms attached to an inert or living surface by a self-produced polymeric matrix or an assemblage of microbial cells associated with a surface enclosed in a matrix of primarily polysaccharide material. A biofilm can comprise any syntrophic consortium of microorganisms in which cells stick to each other and often to a surface. These adherent cells become embedded within an extracellular matrix that is composed of extracellular polymeric substances.

As used herein “multi drug resistant”, “multi drug resistance”, or “multiresistance” refers to a bacteria, biofilm, or other organism that is capable of survival upon exposure to at least one drug from three or more antimicrobial categories.

As used herein “antimicrobial resistance” may refer a bacteria, biofilm, or other organisms that is capable of survival upon exposure to at least one antimicrobial drug.

As used herein “pathogens” or “pathogen” as used herein shall generally mean or encompass viruses, bacteria, fungi, and parasites. In the broadest sense, a pathogen may refer to any organism or agent that may cause or can produce a disease.

As used herein a “toxin” refers to a naturally occurring organic poison produced by metabolic activities of living cells or organisms. For example, a toxin may refer to a substance produced by fungi or bacteria that is growing within the sink drain.

As used herein “bacteria” includes all classes of bacteria and may synonymously refer to microbes. For example, bacteria includes cocci, bacilli, and spirochetes. Additionally, the term “bacteria” refers to both gram positive and gram negative. The term “bacteria” may be used to describe a single bacterium or a population of more than one.

As used herein “eradicating” and “sterilizing” are used synonymously and refers to a decrease in infection-causing microbes by at least a log 5 reduction level.

As used herein “disinfecting” may refer to a decrease in infection-causing microbes by at least a log 4 reduction level.

### Sink Drain Contaminants

Patients may be at risk of catching of hospital-acquired infections from multi-drug resistant organisms that live and grow in the biofilms that can reside in the drains of sinks within hospitals, commercial sinks, and personal sinks. These pathogen growths can thrive and feed off of substances poured down the drain, providing a source of nutrients to allow growth within the drain. The pathogen growth may be capable of becoming aerosolized when the water tap is running. This may result in infections in a subject located in a room with the contaminated sink.

In a first scenario, the pathogens or toxins may be present in hospital sink drains. For example, a bacteria found in the sink drain may be from the family of gammaproteobacterial. The gammaproteobacterial may include the Enterobacteriaceae and Pseudomonas species, and other nonfermenting gram-negative bacilli. These organisms may thrive in wet or moist environments making them a common cause of hospital-acquired infections. In some embodiments, the bacteria may be antimicrobial resistant or multi-drug resistant.

In a second scenario, the pathogens and toxins may be present in a commercial sink or personal sink. For example, a bacteria can include Campylobacter jejuni, Escherichia coli, Salmonella, Staphylococcus aureus, and Legionella. In some embodiments, the bacteria or biofilm located in the sink drain may be ball-shaped, rod-shaped, spirals or helixes or a biofilm comprising any one or combination thereof. The bacteria may be capable of surviving in wet and moist environments making the sink drain a common place for the bacteria to reside, flourish, and/or grow, thus resulting in potential sources for infections and bacterial contaminations. In some embodiments, the bacteria may be Serratia marcescens. A growth of Serratia marcescens, for example, thrives off of consuming phosphorous containing materials or fatty substances such as soap, allowing it to grow in any sink in which a person washed their hands. Thus, shedding light on the resiliency of bacteria to survive even in sinks that may not come in contact with food or drink residues commonly put down a sink.

In both scenarios the pathogen and toxin growth or buildup in the sink drain presents as a source of potential infections for users or subjects located in close proximity to the sink. Other such bacteria that may be present in sink drains can include, but are not limited to Chlamydia, Clostridium, Heliobacterium, Helicobacter, Lactobacillus, Leuconostoc, Listeria, Pediococcus, Shigilla, Vibrio, and Yersinia. In some embodiments, the sink drain may harbor fungi that may result in potential harm to the subjects located in close proximity to the sink. For example, the fungi can include Aspergillus, Penicillium, Saccharomyces and Candida.

### Drain Cover

Described herein are drain covers for sinks to aid in disinfecting, sanitizing, or eradicating microorganism growths that may be present. In some embodiments, the microorganisms may be of a type that cause hospital-acquired infections or infections in subjects that are located in close proximity to a sink that is harboring, or otherwise contaminated, with a microorganism such as a pathogen including a bacteria or fungi.

i. Drain Configurations

Aspects of the present disclosure involve drain covers configured to fit various sink drains as described below. In some embodiments, the sink drain includes hospital sink drains, commercial sink drains, and personal sink drains. For example, the commercial sink drains can include food processing facility sinks and school bathroom or classroom sinks. The sink drain cover described herein may be used on any sink drain currently in use or sink drains currently being tested for use in both public and private markets. One of skill in the art would understand that the sink drain cover described herein may be shaped, sized, or otherwise configured to fit any sink drain.

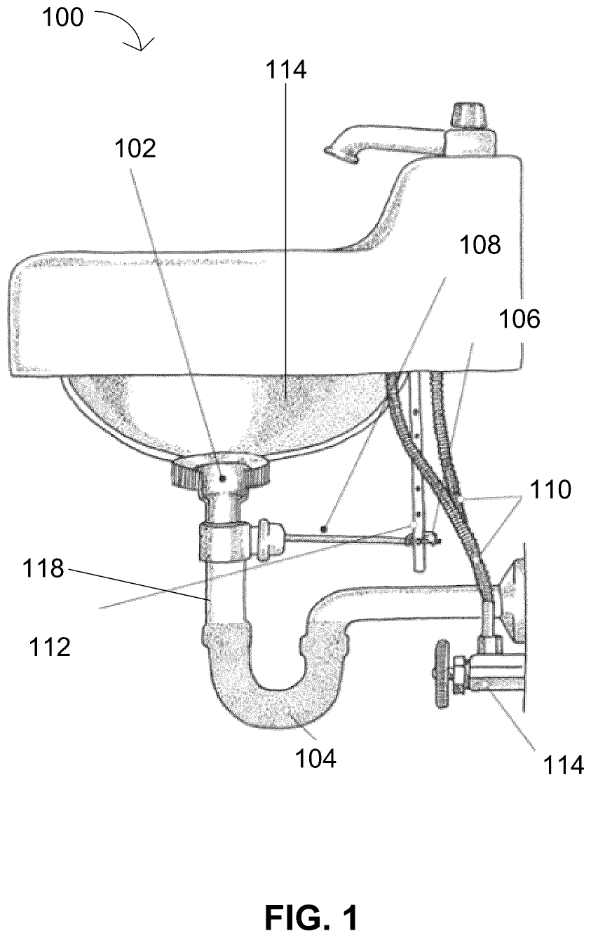

FIG. 1 provides a general schematic of a sink 100 and drainage system, according to one or more embodiments of the present disclosure. It is well understood that while a personal sink is depicted, the figure is used merely as a representative example to highlight various points of the present disclosure. Furthermore, the sink in which the drain cover may be placed or otherwise employed may be any known sink and sink drain in commercial use or residential use. The sink 100 can include a basin 114 that has at least one drain 102 located at the lowest point of the basin 114 and in physical connection with the sink flange and tailpiece 118. The tailpiece 118 may be connected to a trap 104 that may be a p-trap positioned below the sink and designed to maintain a level of water to prevent gas from passing back through the sink and into the room above the sink. Additionally, the p-trap 104 may be replaced or otherwise removed and an S-trap may be used instead. The sink 100 and sink basin 114 may be produced from stainless steel, composite, cast iron, porcelain, copper, glass, natural stone, polymer, or any other suitable material. Additionally, the sink plumbing components, referred to herein as the flange, tailpiece, p-trap, and other plumbing and or piping designed to remove water and contaminants from the sink basin and into the source of water collection, may be comprised of polyvinyl chloride pipes (PVC), metal, other polymer composites such as polypropylene, or any other suitable materials. In some embodiments, the metal may be stainless steel. Other components of a sink may include a clevis 106 and pivot rod 108 for blocking the flow of water down the sink flange and into the tailpiece 118. Additionally, the sink may include a water shut-off valve 114 for turning the supply of water from the water connection point to the sink through tubing 110 and preventing the use of the sink 100.

The sink 100, sink flange or sink drain 102, tailpiece 118 and p-trap 104 may all be locations, or contain surfaces, in which pathogens such as bacteria or fungi can grow. For example, the flange of the sink and the tailpiece may provide surfaces, such as the inner walls of the pipe, where bacteria or fungi can attach and grow. The growth of the bacteria may be dependent upon the type and the environmental conditions in which the bacteria are exposed. For example, bacteria such as E. coli, salmonella, shigella, campylobacter, norovirus, and hepatitis A. may be commonly found in the kitchen sink and bathrooms in homes and other public places. These bacteria may survive on a surface from a few hours to months, giving ample time for potential subject exposure to occur. Bacteria located in a sink drain or in the plumbing connected to the sink may be aerosolized as water passes through the sink, down the drain, and contacts the water in the p-trap 104 causing a small splash to occur.

In hospital sinks, bacteria can include the Enterobacteriaceae and Pseudomonas species, and other nonfermenting gram-negative bacilli. These organisms may thrive in wet or moist environments making them a common cause of hospital-acquired infections. In some embodiments, the bacteria may be antimicrobial resistant or multi-drug resistant. In the right conditions, the bacteria may be capable of surviving for up to four weeks. The long life-span of these bacteria presents many problems for patients and visiting family in hospitals and hospital staff. In some embodiments, the bacteria may grow to produce a biofilm within the plumbing and sink drains. Biofilms may live for years within the sink drain and may be difficult to remove.

Current methods for eradicating or otherwise disinfecting and or sanitizing a sink and drain or drain plumbing includes methods such as pouring chemicals down the drain, replacing drain plumbing, replacing the sink or installing devices along the sink drain and plumbing to combat the growth of bacteria. Current methods may not be effective in decontaminating, disinfecting, or sterilizing the sink. For example, pouring chemicals including bleach, acetic acid, or hydrogen peroxide may not be effective at eradicating or disinfecting the sink drain and bacteria may grow back after treatment. Furthermore, the concentration of chemicals or amount needed to reduce, eradicate, render inert, or non-infectious of the pathogen or the toxin may be non-environmentally friendly. Alternatively, replacing components of the sink may be costly and time inefficient. Additionally, placing devices along the sink drain pipes, including devices that heat up the piping, may be a source of potential fire hazard making some solutions unsafe for practical everyday use. Thus, new methods for killing, destroying, and/or rendering inert or non-infectious the pathogen and/or the toxins residing in sink and sink drains or drain plumbing are needed.

ii. Device Configurations

Aspects of the present invention involve a device for placement in or over a sink drain designed for disinfecting, eradicating, sanitizing, killing, destroying, and/or rendering inert or non-infections a pathogen and/or toxin. The pathogen can include bacteria, fungi, or a combination thereof, and a toxin can include a composition generated by bacteria, fungi, or combination thereof. Of course, it should be understood that, in embodiments, both the pathogen(s) and toxin(s) may be present. The device described herein may have multiple different configurations or parts that may be removed or replaced to increase the longevity of the device in use. For example, the device may have a removable battery unit that can be replaced after the life cycle of the original battery is depleted. The device described herein may be an environmentally friendly device for disinfecting, sanitizing, killing, destroying or rendering inert the pathogen or toxin within the sink and sink drain.

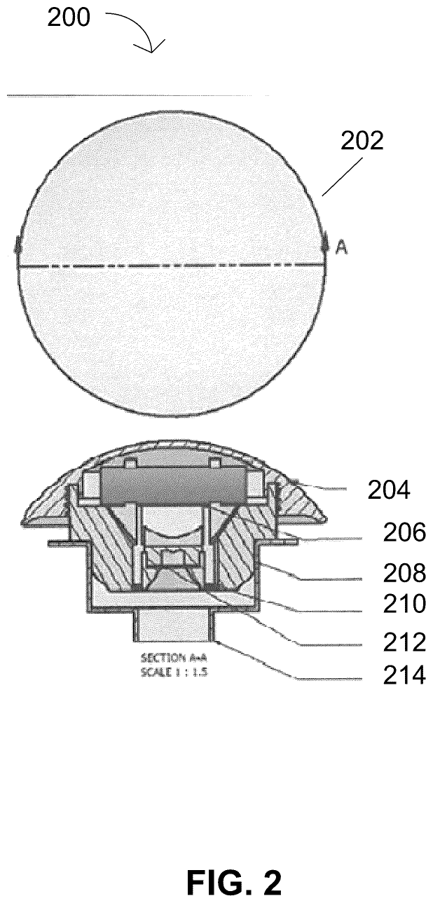

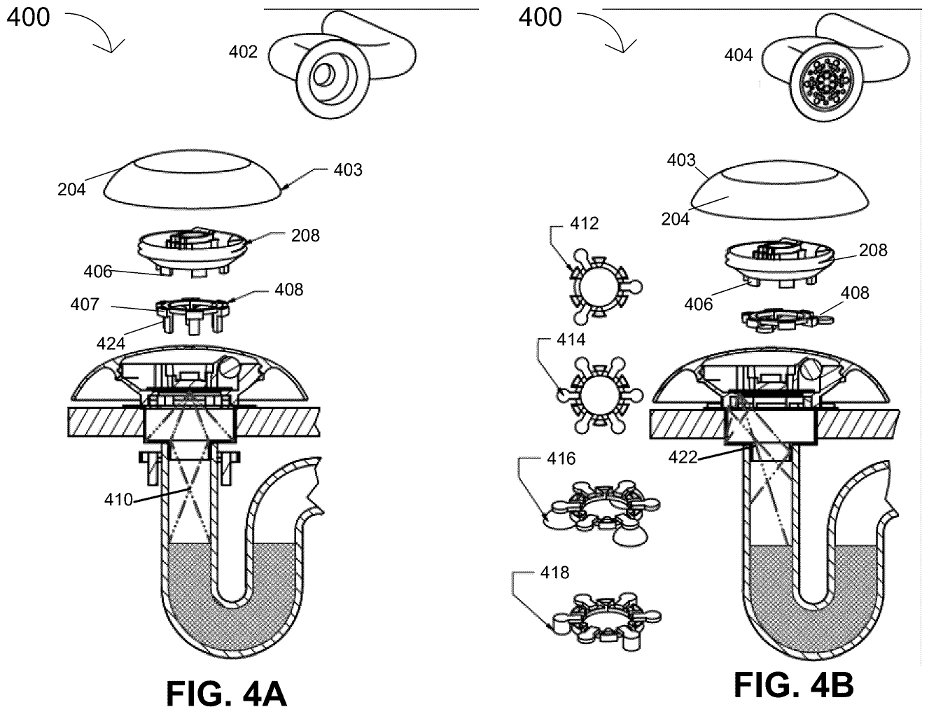

FIG. 2 provides an illustration of an example schematic for a sink drain cover device 200, according to some embodiments of the present disclosure. The device 200 may comprise a housing that includes a top component, also referred to as a first component, 204 and a bottom component, also referred to as a second component 208. The housing may be configured for enclosing the electronic components such as the electronic circuit and at least partially enclosing the light source. The top component 204 and the bottom component 208 may be configured to be replaceable, such as if one of the top component 204, the bottom component 208, or both are damaged or destroyed, while the internal components still remain useful. For example, if a chemical is poured onto the top of the device that may cause the device to become contaminated, deformed, or otherwise rendered unusable, the top component 204 of the housing may be removed from the device 200 and a new top component 204 may be attached to the bottom component 208. In some embodiments, the bottom component 208 may be replaceable with a different bottom piece 208 that has a different configuration than the first bottom component 208. For example, if the sink drain 214 has a recessed section, the bottom component used may protrude, or fit into the sink recess near the drain 214 such that the light from the light emitting diode (LED) is directed down the sink drain 214 and towards the p-trap. The configuration may be illustrated more clearly in FIGS. 4A and 4B. In another example, if the sink drain 214 does not have a recessed drain, the bottom component of the housing may be replaced with an alternate bottom component such that the bottom no longer protrudes down the drain but sits flush with the sink drain. In some embodiments, the second component (bottom component) may be designed to fit over a protruding sink drain 214. In all configurations, the device 200 may be configured to allow water to pass between the device 200 and the sink drain 214, flowing into the plumbing of the sink. In embodiments, when the top component 204 and the bottom component 208 are in physical connection to one another, the device is air and water-tight. For example, the device internal components, within the housing, may not come in contact with a liquid when the device is placed within the sink drain. In some examples, the device 200 may comprise a single housing component (e.g. the top component 204 and bottom component 208 are formed from a unitary component), such as that is adapted or fit around the internal components.

The top component 204 of the device 200 may be configured to reduce or minimize splashing of water upon contact, such as compared to conventional sink drain components. For example, the top component 204 may have a convex shape such that the convex shape is positioned directly under the sink faucet. When water is flowing, the water stream may come in contact with the top of the top component 204 such that the water is dispersed across the surface of the device and splashing is reduced, minimized, or eliminated. The top component 204, may be a circular shape 202 and have a diameter that is larger than the sink drain opening such that 100% of the sink drain opening is below the top component, limiting any water from directly flowing from a faucet into the trap causing splashing and aerosolization of pathogens or toxins within the trap. For instance, in embodiments, the diameter of the top component 204 may have a diameter greater than or about 2.5% larger than a diameter of a sink drain opening, such as greater than or about 5%, greater than or about 7.5%, greater than or about 10%, greater than or about 12.5%, greater than or about 15%, greater than or about 17.5%, greater than or about 20%, or any ranges or values therebetween.

In some embodiments, the device 200 may be made from, formed from, or otherwise produced from, a plastic material, a urethane material, a metal material, or one or more other materials, as well as combinations thereof. The material selected may be water repellant, water resistant, or water-proof. In some embodiments, the material selected may have antimicrobial properties. For example, the device 200 may be made from a thermoplastic or a thermosetting plastic, or combinations thereof. In embodiments, the device 200 may be made from acrylonitrile butadiene styrene (ABS), polylactic acid (PLA), polyethylene terephthalate glycol (PETG), nylon, thermoplastic polyurethane (TPU), polyvinyl alcohol (PVA), high impact polystyrene (HIPS), a silver-infused polymer, a copper-infused polymer, a quaternary ammonium compounds (QACs)-infused polymer, a zinc oxide (ZnO)-infused polymer, a titanium dioxide (TiO2) infused polymer, a UV reactive photocatalytic coating (alone or in combination with a further polymer), a stainless steel, a composite material, or combinations thereof. In some embodiments, the device 200 may be made from a resin. For example, the resin may be a standard resin, a clear resin, a draft resin, a rigid resin, a polyurethane resin, a high temperature resin, a flexible and/or elastic resin, a silicone resin (including silicon 40A resins), a medical and/or dental resin, a flame-retardant resin, or combinations thereof. Other materials that may be used for producing the device 200 may include nylon 12, nylon 11, nylon composites, polypropylene, or combinations thereof. It will be appreciated that other materials may be used for the devices described herein.

Device 200 includes a charging coil 210, an energy storage device 206, and at least one light emitting diode (LED) 212. The LED may be positioned in the housing for directing the light into the tailpiece and to the trap of the drain for killing, destroying, and/or rendering inert or non-infectious pathogens and/or toxins therein or thereon. In some embodiments, the device may include one light emitting diode positioned inside the housing for directing the light down the tailpiece and into the p-trap. In some embodiments, the device may include more than one, for example 2, 3, 4, or 5 LEDs, or more, positioned in the bottom piece of the device and within the housing for directing light down the tailpiece and into the p-trap. For example, the LEDs may be spaced out equally along the bottom piece of the device to direct light over at least 90% of the surface area below the device. The LED may be any LED that is configured for generating a light capable of and/or configured for killing, and/or rendering inert or non-infectious a pathogen and/or toxin. In some embodiments, the charging coil 210, may be positioned or built into a printed circuit board (PCB) and placed within the bottom component 208. Additionally or alternatively, in embodiments, the charging coil 210 may be formed separately from a PCT or other electronic component, and placed near the PCB and/or electronic component on a side of the PCB board containing one or more LEDs. In such a configuration, the adapter 408 and 420 (see, e.g., FIGS. 4A and 4B) may be first removed from the bottom component 208 before the device is charged on the charging pod.

The LED can include, for example, an LED that emits light in the wavelength range of from 180 nm to 400 nm. For example, an LED capable of emitting light in a wavelength range of from 200 nm to 280 nm may be effective in killing microorganisms. However, other wavelengths and ranges thereof may be utilized depending upon the target microorganism, fungi, and/or toxin thereof. It may be understood by one skilled in the art that light within the UV wavelength range is capable of being absorbed by the bacteria and into the nucleic acids causing DNA abnormalities. The light-induced damage to the DNA and RNA of a microorganism often results from the dimerization of pyrimidine molecules. In particular, thymine (which is only found only in DNA) produces cyclobutane dimers. When thymine molecules are dimerized, it becomes very difficult for the nucleic acids to replicate and if replication does occur it often produces a defect that prevents the microorganism from being viable. In some embodiments, the process may be referred to as ultraviolet germicidal irradiation when at a wavelength of from 180 nm to 280 nm.

The wavelength of light may be varied in the device by use of one or more alternate LEDs that emits light of one or more different wavelengths. In some embodiments, the device, may include multiple LEDs, where some or all of the LEDs include LEDs that emit light at different wavelengths across the entire spectrum of UV light. For example, one LED may emit light at a wavelength of from 180 to 280 nm and a second LED may emit light in a range of from 280 nm to 315 nm. UV light may be divided into three separate bands based upon their wavelength (e.g., UVA (315 nm to 400 nm), UVB (280 nm to 315 nm), and UVC (100 nm to 280 nm)). In some embodiments, the device may be configurable to replace or otherwise change the LED within the housing. In an alternate embodiment, dual wavelength LED configurations may include combining an LED that emits light at a wavelength of about 215 nm to about 230 nm, such as about 220 nm to about 225 nm, or such as about 222 nm, in embodiments, and a second LED that may emit light at about 395 nm to about 415 nm, such as about 400 nm to about 410 nm, or such as about 405 nm, or any ranges or values therebetween. In some embodiments, the dual LED configuration may be more effective at disinfecting or eradicating bacteria from the sink drain than either wavelength alone (e.g. may exhibit a synergistic effect). Light at a lower wavelength for instance, may be beneficial because it may not exhibit the effects to skin than the more common 270-279 nm range. In some embodiments, the dual platform LED may emit light at a wavelength of from 180 nm to about 250 nm and the second wavelength LEDs within the device may emit light from about 315 nm to about 450 nm. In embodiments, one or more wavelengths may be selected in order to produce, or increase production of, ozone from ambient oxygen, which may further improve the disinfecting or eradication of bacteria or fungi from the sink drain.

As mentioned above, the LED may emit light in the UV wavelength range of from 180 nm to 400 nm. For example, the UV light emitted may be 180 nm to 200 nm, from 200 nm to 280 nm, from 280 nm to 315 nm, or from 315 nm to 400 nm. In some embodiments, the LED may be a wavelength specific LED. For example, most commercial LEDs may be centered around a specific wavelength ±10 nm. Thus, it may be understood that an LED falling within the wavelength ranges above may be used within the devices described herein. The light emitted from the LED that is positioned in the bottom of the housing may direct light at an angle of from 0° to 60° relative to a wall of the tailpiece. For example, the LED may emit light at an angle of from 0°, 2°, 4°, 6°, 8°, 10°, 12°, 14°, 16°, 18°, 20°, 22°, 24°, 26°, 28°, 30°, 32°, 34°, 36°, 38°, 40°, 42°, 44°, 46°, 48°, 50°, 52°, 54°, 56°, 58°, 60°, any angle between, or including a range of angles as an array relative to a wall of the tailpiece.

In some embodiments, the LED may have a power consumption or an average radiant flux of 0.01 W to 5 W. For example, the power consumption or average radiant flux may be 0.01 W, 0.02 W, 0.03 W, 0.04 W, 0.05 W, 0.06 W, 0.07 W, 0.08 W, 0.09 W, 0.10 W, 0.2 W, 0.3 W, 0.4 W, 0.5 W, 0.6 W, 0.7 W, 0.8 W, 0.9 W, 1.0 W, 1.2 W, 1.4 W, 1.6 W, 1.8 W, 2.0 W, 2.2 W, 2.4 W, 2.6 W, 2.8 W, 3.0 W, 3.2 W, 3.4 W, 3.6 W, 3.8 W, 4.0 W, 4.2 W, 4.4 W, 4.6 W, 4.8 W, 5.0 W, or any combination thereof. In some embodiments, the device may be configured such that no two LEDs are emitting light at the same time. In yet another embodiment, the device may be capable of having more than one LED emitting light at any singular time.

In embodiments, the LED may be described based upon the radiant intensity of the LED. The LED described herein may have a radiant intensity of from 0.100 mW/cm² to 0.500 mW/cm². For example, the radiant intensity may be 0.100 mW/cm², 0.110 mW/cm², 0.120 mW/cm², 0.130 mW/cm², 0.140 mW/cm², 0.150 mW/cm², 0.160 mW/cm², 0.170 mW/cm², 0.180 mW/cm², 0.190 mW/cm², 0.200 mW/cm², 0.210 mW/cm², 0.220 mW/cm², 0.230 mW/cm², 0.240 mW/cm², 0.250 mW/cm², 0.260 mW/cm², 0.270 mW/cm², 0.280 mW/cm², 0.290 mW/cm², 0.300 mW/cm², 0.310 mW/cm², 0.320 mW/cm², 0.330 mW/cm², 0.340 mW/cm², 0.350 mW/cm², 0.360 mW/cm², 0.370 mW/cm², 0.380 mW/cm², 0.390 mW/cm², 0.400 mW/cm², 0.410 mW/cm², 0.420 mW/cm², 0.430 mW/cm², 0.440 mW/cm², 0.450 mW/cm², 0.460 mW/cm², 0.470 mW/cm², 0.480 mW/cm², 0.490 mW/cm², 0.500 mW/cm², or any intermittent value or combination thereof.

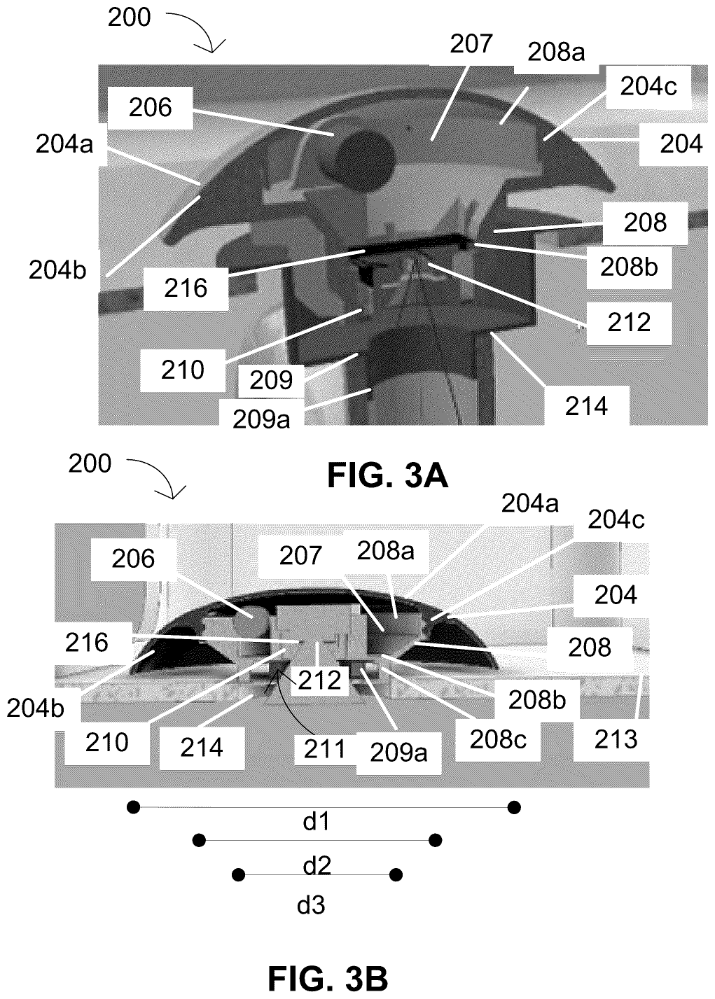

FIG. 3A and FIG. 3B provide various illustrations of example diagrams for sink drain covers. FIG. 3A provides an illustration of an exemplary sink drain cover configured to fit within a recessed drain of a sink, and FIG. 3B provides an illustration of an exemplary sink drain cover configured to fit over a non-recessed drain of a sink, according to some embodiments of the present disclosure. However, it should be clear that features of the exemplary sink drain covers may be interchangeable, as discussed in greater detail below.

In embodiments, the top component 204 may include a top surface 204a and an underside 204. As illustrated, in embodiments, the top surface 204a may exhibit a convex or domed shape, in order to control splashing or dropping of a liquid. However, in embodiments, the top component may have other profiles, including flat, circular, disk, or the like. Nonetheless, as illustrated, in embodiments, the top component may have a top component diameter d1 (shown more clearly in FIG. 3B). In embodiments, the diameter of the top component may be selected so as to fully cover a sink drain. Thus, in embodiments, the diameter d1 may be greater than or about 2.5% larger than a diameter of a sink drain opening, such as greater than or about 5%, greater than or about 7.5%, greater than or about 10%, greater than or about 12.5%, greater than or about 15%, greater than or about 17.5%, greater than or about 20%, greater than or about 25%, greater than or about 30%, greater than or about 35%, greater than or about 40%, greater than or about 45%, greater than or about 50%, greater than or about 55%, greater than or about 60%, greater than or about 65%, greater than or about 70%, greater than or about 75%, greater than or about 80%, greater than or about 90%, greater than or about 100% larger, or any ranges or values therebetween.

Moreover, in embodiments, the component may include one or more mounting brackets 204c formed on the underside 204b. In embodiments, the mounting bracket 204c may be a unitary piece that extends in a direction generally orthogonal to underside 204b which may be a vertically extending direction, in embodiments. For instance, in embodiments, the one or more mounting brackets 204c may extend from the underside, in a direction generally opposite the top surface and/or towards a sink drain 214. Moreover, in embodiments, the one or more mounting brackets 204c may be a single mounting bracket that extends circumferentially around the selected portion of the underside (e.g. around a circumference of the top component, or at a location spaced laterally inward from the exterior circumference, discussed in greater detail below). In such a manner, the mounting bracket may interact with a corresponding mated piece on the bottom component, to form a liquid tight seal at the interface of the top component and the bottom component.

In embodiments, the one or more mounting brackets may be formed at a diameter d2 that is smaller than the diameter of the top component d1 (shown more clearly in FIG. 3B). For instance, in embodiments, the mounting bracket location may be at a diameter d2 that is greater than or about 5% less than the top component diameter d1, such as greater than or about 10%, greater than or about 12.5%, greater than or about 15%, greater than or about 17.5%, greater than or about 20%, greater than or about 25%, greater than or about 30%, greater than or about 35%, greater than or about 40%, greater than or about 45%, greater than or about 50% less, or any ranges or values therebetween. In such a manner, the top component may extend past or overhang bottom component 208 and/or drain adapter 209. Furthermore, as will be discussed in greater detail below, by forming mounting bracket(s) 204c at a location corresponding to a smaller diameter than the overall diameter of the top component, the internal volume 207 formed between the top component and the bottom component may be further protected from flowing or splashing liquid.

Nonetheless, in embodiments, the device may also include a bottom component 208, which may be permanently or releasably affixed to top component 204. For instance, in embodiments, the bottom component 208 may have a top surface 208a and a bottom surface 208b. In embodiments, top surface 208a may have a size generally corresponding to diameter d2 discussed above. Thus, in embodiments, top surface 208a may interact with mounting bracket(s) 204c in order to affix the bottom component 208 to the top component 204. In embodiments, the top surface 208a and mounting bracket(s) 204c may have a complementary male/female design, such as a snap-fit, screw-fit, and/or lock in place, as well as other releasable and permanent connections.

In embodiments, the bottom surface 208b may have a diameter d3 that is less than the first diameter d1 and/or the second diameter d2. In embodiments, the lower surface diameter d3 may be approximately equal to a diameter of a sink drain 214. In embodiments, the lower surface diameter d3 may be greater than or about 5% less than diameter d2, such as greater than or about 10%, greater than or about 12.5%, greater than or about 15%, greater than or about 17.5%, greater than or about 20%, greater than or about 25%, greater than or about 30%, greater than or about 35%, greater than or about 40%, greater than or about 45%, greater than or about 50% less, or any ranges or values therebetween. Thus, in embodiments, a sidewall of the bottom component may have a sloped orientation between the top surface 208a and bottom surface 208b, but other orientations are also considered herein.

Furthermore, in embodiments, the bottom surface 208b may include one or more supports 208c. In embodiments, such as the flush drain illustrated in FIG. 3B, the supports 208c may contact a surface of the sink or drain in order to maintain a gap between the bottom surface 208b and the sink or drain surface. However, in further embodiments, the supports 208c may be utilized to releasably or permanently affix the bottom component to a drain adapter 209. Regardless of the use of supports 208c, in embodiments, the bottom surface 208b may define an aperture 211. In embodiments, the aperture 211 may be centrally located (e.g. around an approximate center point of the diameter), but may be located in an offset orientation depending upon the drain to be covered. As illustrated, the aperture 211 defines an opening in the bottom component, allowing the LEDs 212 to emit into the drain 214 while protecting the LEDs in the central volume 207, reducing the risk or likelihood of splashing while allowing for full cleaning capabilities. Furthermore, as illustrated, the shape and size of the top component and the bottom component a central volume 207 therebetween that may be occupied by one or more components that will be discussed in greater detail below.

In embodiments, the device may also include a drain adapter 209. For instance, in embodiments, the supports 208c may be inadequate for maintaining the device above certain drain configurations, such as a recessed drain. Thus, in embodiments, the supports 208c may interact with complementary receptors (shown more clearly in FIGS. 4A and 4B) to permanently or releasably affix the bottom component 208 to the drain adapter 209. However, in embodiments, it should be understood that the drain adapter 209 may be formed unitarily with bottom component 208. Nonetheless, the drain adapter may have a size approximately equal to the diameter of the bottom surface 208b, such as d3 in the illustrated embodiment. However, the drain adapter 209 may have a larger or smaller diameter than lower surface diameter d3, to accommodate the connection and the drain, in embodiments. Nonetheless, in embodiments, drain adapter 209 may define a central aperture that corresponds in size and shape to the central aperture defined by the bottom component, in order to maintain the opening for line of sight access of the LED to the drain 214. Nonetheless, in embodiments, the drain adapter 209 may include one or more legs 209a that extend in a generally vertical direction, that may be generally orthogonal to bottom surface 208b, that support the bottom surface 208b above a surface of the sink 213 that supports drain 214. As discussed in greater detail in regard to FIGS. 4A and 4B, in embodiments, the one or more legs 209a may be discrete and spaced apart legs 209a, or may be a unitary piece that extends circumferentially around aperture 211. Furthermore, while the present specification has so far referred to diameters and other circular dimensions, it should be clear that such terms may refer to non-circular shapes, and thus may also include equivalent diameters.

The device 200 can include an energy storage device 206, positioned within the central volume 207 of the housing for powering the device 200. For purposes of the description provided herein, an example of an energy storage device may be a battery. Thus, throughout the present disclosure, an energy storage device may be referred to as a battery. One skilled in the art may understand that any alternate energy storage device capable of fitting within the sink drain device may be employed. The battery may be in electrical connection with an electronic circuit also positioned within the housing for powering the device 200. In some embodiments, the battery may be removed and replaced. For example, to provide a device as described herein capable of killing, disinfecting, or sterilizing a drain in a cost-effective manner, the battery may be replaced without requiring replacement of the device as a whole. The replacement of a battery may increase the lifespan of the device, thus providing an economically friendly solution for preventing pathogen or toxin growth within the sink and drain. In some embodiments, the battery is a standard or commercial rechargeable battery.

The device 200 may also include an electronic circuit board 216 (which may be or include the PCB discussed above) that is configured to and/or arranged to control emission of light from the LED 212. The electronic circuit board 216 may be disposed within the central volume, and may be seated on a mounting surface within the central volume, or may be molded to one or more internal surfaces thereof. The electronic circuit may include a multitude of components for operating and using the device. For example, the electronic circuit 216 may be in electrical connection with the energy storage device and a charging circuit for receiving energy to charge the energy storage device. The charging circuit may include an induction coil for wirelessly receiving energy to charge the energy storage device or a wired charging port for receiving energy to charge the energy storage device. The port for charging may be positioned within the housing of the device such that when the device is used, water does not enter the internal compartment of the device.

As illustrated, in embodiments, the LEDs 212 may be formed in a surface of the electronic circuit board 216, and therefore also disposed in the central volume. In embodiments, the LEDs 212 may be formed in a lower surface of the electronic circuit board 216 such that the emitted light from the LEDs 212 is directed towards central aperture 211 (and therefore drain 214). In embodiments, the surface of the electronic circuit board 216 containing LEDs 212 may be disposed adjacent to central aperture 211 so as to be protected by bottom surface 208b while maintaining line of sight access through the central aperture 211. Nonetheless, in embodiments, the surface of the electronic circuit board 216 containing LEDs 212 may be disposed adjacent to a vertical midpoint, or higher, within the central volume 207, and the direction of the emitted light may be controlled by path control mechanisms, such as mirrors.

The electronic device may also include an indicator for communicating information about the device or electronic circuit. For example, the indicator may be an audible indicator, a visual indicator, or a wireless signal including the information about the device or electronic circuit. The information about the device may include, among other things, a battery charge state, an orientation of the device, or an operation error of the device.

In some embodiments, the device may also include an inertial sensor, an orientation sensor, a position sensor, or a combination thereof, disposed on bottom component 208. For example, the device may include an inertial sensor that may cut off or be configured to cut off power to the LED when the device is positioned upside down (e.g., when the portion of the electronic circuit containing the one or more LEDs is facing vertically upward, opposite a direction of the sink 213). The inertial device may be an added safety feature within the device to prevent undesirable UV exposure. An inertial sensor may relay information to the electronic circuit relating to a movement of the device. In embodiments, the device may include an orientation sensor or a position sensor as an addition or alternate to an inertial device. In some embodiments, the device may include all three sensors within the device for relaying a movement of the device, an orientation of the device, and a position of the device. In some embodiments, the inertial sensor may be used to perform the operations of turning on the LED when the sink is actively in use or liquid is flowing on or around the device, as the inertial sensor detects the flow of a liquid. In such an operation, the LEDs may neutralize any pathogens that might become aerosolized by water flowing into the sink drain (e.g., preventing the aerosolized pathogens travel upwards from the p-trap or tailpipe towards the sink drain).

FIG. 4A and FIG. 4B provide various illustrations of example diagrams for sink drain covers. FIG. 4A provides an illustration of an exemplary sink drain cover configured to fit within a recessed drain 402 of a sink. The device 400, may include a top component 204 and a bottom component 208 that may be physically connected (releasably or permanently) such that when the top compartment 204 and the bottom component 208 are connected, they are water tight, such as utilizing any of the orientations discussed above in regard to FIGS. 3A and 3B. In some embodiments, the top component 204 may be or include a convoluted splash path attachment 403. For example, a convoluted splash path attachment 403 may include a convex shape on the top portion of the convoluted splash path attachment 403. When water hits the convoluted splash path attachment 403, the water may not splash back up towards the operator or user of the sink or may not cause water droplets to hit the side walls of the sink.

In embodiments, the bottom component 208 may include supports 406, which may correspond to supports 208c discussed above, positioned along the bottom surface of the bottom component 208. The supports 406 may be fabricated, or otherwise included to allow a drain adapter 408 to be connected to the bottom portion of the bottom component 208. In embodiments, supports 406 may be releasably or permanently retained in corresponding recesses 407 in drain adapter 408. In embodiments, the supports 406 may exhibit a snap fit relationship with recesses 407, as well as other connections as known in the art.

In addition, as illustrated, the recessed drain 402 main exhibit a depth that is greater than a height of supports 406. In such embodiments, a drain adapter 408 may therefore be utilized to support the device above a surface of the sink. In embodiments, the drain adapter 408 may have legs 424 positioned extending away from the drain adapter body, such that when the adapter 408 is positioned into the recessed sink drain 402, the legs 424 of the drain adapter 408 may be in physical contact with the sink drain 402. In embodiments, as discussed above, the diameter of the drain adapter 408 (and/or portion thereof containing legs 424) may be approximately the same as a diameter of a drain, or smaller, so as to apply an outward pressure on the sink drain 402, thus maintaining the sink drain cover positioned over the sink drain 402. In some embodiments, the drain adapter 408 may have 3 legs 424 as illustrated by drain adapter 412 or may have 6 legs 424, as illustrated by the sink adapter 414. As illustrated, legs 424 may help to releasably secure the drain adapter 408 within the sink drain 402, maintaining orientation of the LEDs over the drain and trap. Additionally, the drain adapter 408 may be capable of having multiple types of connections to the sink drain 402. For example, the drain adapter 408 may have or be configured to have suction cups 416 positioned along a bottom surface of the drain adapter 408 for adhering the sink drain cover above the sink drain. In another example, the drain adapter 408 may have or be configured to have magnets 418 positioned along the bottom surface of the sink adapter 420 for adhering the sink drain cover above the sink drain. However, it should be clear that other permanent or releasable attachments may be utilized.

FIG. 4B provides an illustration of an exemplary sink drain cover configured to fit over a non-recessed, or flush, drain 404 of a sink, according to some examples of the present disclosure. However, it should be clear that features of the exemplary sink drain covers may be interchangeable, and that the device discussed herein may include any one or more of the features discussed above. The device 400, may include a top component 204 and a bottom component 208 that may be physically connected such that when the top compartment 204 and the bottom component 208 are connected, they are watertight, such as utilizing any of the orientations discussed above in regard to FIGS. 3A and 3B. In some embodiments, the top component 204 may be, or be physical connected with, a convoluted splash path 403 attachment as discussed above. For example, a convoluted splash path attachment 403 may exhibit a convex shape on the top portion (e.g. externally facing portion) of the convoluted splash path attachment 403. When water hits the convoluted splash path attachment 403, the water may not splash back up towards the operator or user of the sink or may not cause water droplets to hit the side walls of the sink.

In embodiments, the bottom component 208 may include supports 406 positioned along the bottom of the bottom component 208. The supports 406 may be fabricated, or otherwise included to provide for a drain adapter 408 to be connected to the bottom portion of the bottom component 208, as discussed above. In some embodiments, a sink adapter 420 may be a 3 legs as illustrated in drain adapter 412 or a 6 legs as illustrated by drain adapter 414, however, more or less point of connection are contemplated herein for drain adapter 408, as well as any ranges or values therebetween. Additionally, the drain adapter 408 may be capable of having multiple types of connections to the sink drain 404. For example, the drain adapter 408 may have or be configured to have suction cups 416 positioned along a bottom surface of the drain adapter 408 for adhering the sink drain cover above the sink drain. In another example, the drain adapter 408 may have or be configured to have magnets 418 positioned along the bottom surface of the drain adapter 408 for adhering the sink drain cover above the sink drain. However, it should be clear that other permanent or releasable attachments may be utilized.

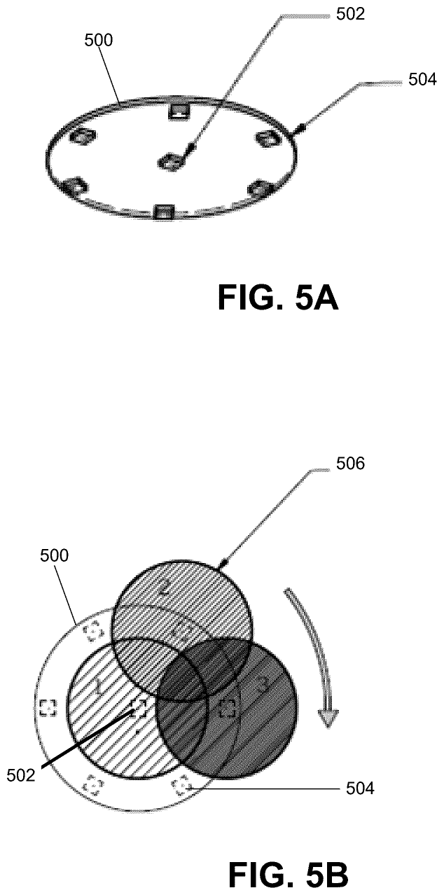

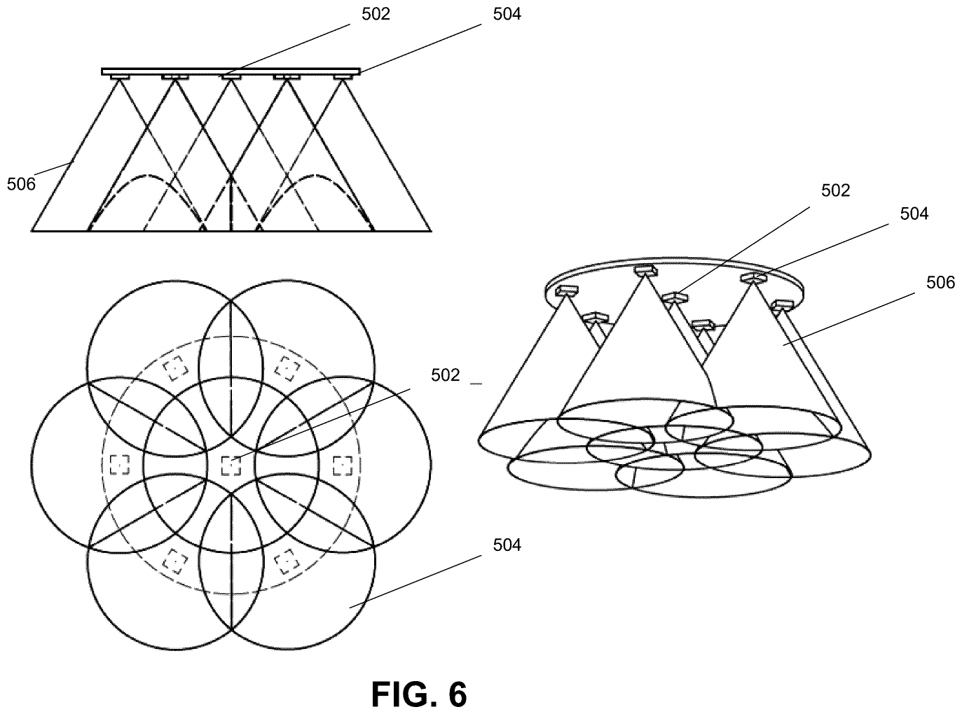

FIG. 5A and FIG. 5B provide illustrations of example LED configurations for sink drain covers. FIG. 5A provides an illustration of an exemplary configuration of LEDs positioned on the bottom components of the sink drain cover, and FIG. 5B provides an illustration of exemplary light emitted from the LEDs positioned along the bottom component of the sink drain, according to some examples of the present disclosure. However, it should be clear that features of the exemplary sink drain covers may be interchangeable. In some embodiments, an electronic circuit board 500 included in the bottom component 408 may include a first surface, and a second surface including one or more LEDs 502, 504. In embodiments, a single LED 502 may be placed at an approximate center point of the PCB 500, or may include more than one led positioned in a concentric circle around an exterior of the electronic circuit, or surrounding the single LED, as well as other orientations. In embodiments, the LED configuration may include a central LED 502 and a series of concentric LEDs 504 positioned around the central LED 502, such as around an exterior of PCB 500. The LEDs may emit light in a cone shape 506 (shown more clearly in FIG. 6) irradiating outward away from the LED and down the sink drain as depicted in FIGS. 4A and 4B. However, it should be clearly that other emitted light shapes are contemplated herein.

In embodiments, the LEDs may emit light simultaneously in a continuous mode or a sequence (e.g. intermittent or pulsing) mode. In some embodiments, the LEDs may emit light in a single sequence array. For example, in a single sequence array, a first LED may emit light while the others are off for a period of time followed by the first light shutting off and a second LED turning on for a period of time (e.g., individual LEDs operating in a pre-selected sequence). The process is repeated until each LED has operated in an on manner for a period of time. The time the LEDs are on may be sufficient enough to kill at least 95% of bacteria within the sink drain. In embodiments, a LED 502 disposed an approximate center of circuit 500 may emit light according to the ray tracing 410 shown in FIG. 4A. In embodiments, the light emitted from one or more of the LEDs 504 extending around a perimeter of the circuit 500 may emit light according to the ray tracing 422 shown in FIG. 4B.

FIG. 6 provides various illustrations of exemplary LED light emission cones from the LED configurations discussed herein. For example, the light emitted from the LEDs may overlap with one another, in embodiments. The light overlapping and irradiating from all of the LEDs may provide sufficient coverage of the sink drain including shadow areas of the sink drain that may be present from a sink drain mesh for preventing food or large debris from falling into the sink. The use of multiple LEDS may allow for light to pass through areas in which a single drain LED might not reach, further improving the sterilization properties of the drain covers discussed herein.

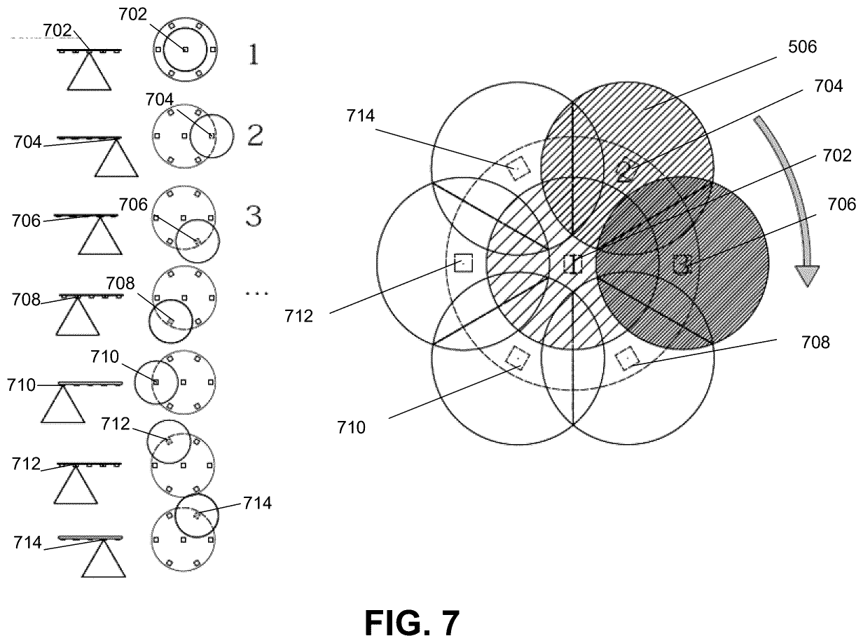

FIG. 7 provides an illustration of an example array sterilization sequence of the LEDs, according to some examples of the present disclosure. The sequence of light may first start with the central LED 702. The central LED 702 may emit light for a predetermined mount of time before shutting off, and the second LED 704 turns on. The process may be repeated until each LED in the series performs the action of turning on. For example, after the second LED 704 emits light, the second LED 704 may turn off and the third LED 706 may turn on. The sequence may be performed 1 time or multiple series. Each sequence of all seven LEDs turning on and then shutting off may be considered a series. For example, the LED configuration may be operated in 1, 2, 3, 4, 5, 6, 7, 8, 9, or 10 series. In some embodiments, there may be more than 10 series performed to clean the sink drain. However, it should be clear that, in embodiments, more or less LEDs may be utilized, as well as that alternating and various sequences are contemplated herein. In addition, as discussed above, in embodiments, continuous emittance may be utilized, as well as where two, three, four, or more LEDs are utilized simultaneously.

In some embodiments, the device may also include a wireless transceiver (not shown), such as for communicating wirelessly with a mobile device, handheld device, or computer system. In some embodiments, the device may comprise a Wi-Fi-enabled microcontroller or a Bluetooth enabled microcontroller. The Wi-Fi controller or Bluetooth microcontroller may be employed for controlling the device from a remote location. In some embodiments, the microcontrollers may be employed for relaying information from the device wirelessly to a hand-held device or a computer. For example, a hand-held device can include a smartphone.

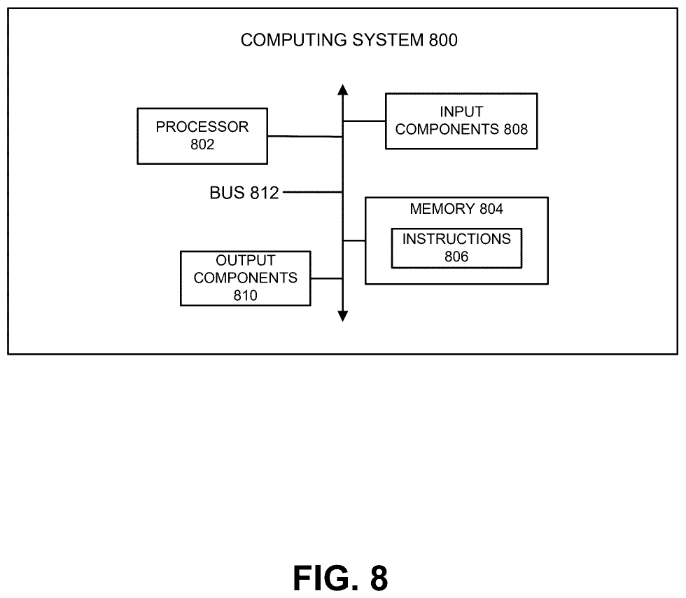

FIG. 8 provides a schematic of a block diagram of an example of a computing device usable for implementing some embodiments of the present disclosure. The computing device 800 includes a processor 802 coupled to a memory 804 via a bus 812. The processor 802 can include one processing device or multiple processing devices. Examples of the processor 802 include a Field-Programmable Gate Array (FPGA), an application-specific integrated circuit (ASIC), a microprocessor, or any combination of these. The processor 802 can execute instructions 806 stored in the memory 804 to perform operations. In some examples, the instructions 806 can include processor-specific instructions generated by a compiler or an interpreter from code written in any suitable computer-programming language, such as C, C++, C#, Python, or Java.

The memory 804 can include one memory device or multiple memory devices. The memory 804 may be non-volatile and include any type of memory device that retains stored information when powered off. Examples of the memory 804 can include electrically erasable and programmable read-only memory (EEPROM), flash memory, or any other type of non-volatile memory. At least some of the memory 804 includes a non-transitory computer-readable medium from which the processor 802 can read instructions 806. A computer-readable medium can include electronic, optical, magnetic, or other storage devices capable of providing the processor 802 with computer-readable instructions or other program code. Computer-readable storage media includes, but is not limited to, RAM, ROM, erasable programmable ROM (“EPROM”), electrically-erasable programmable ROM (“EEPROM”), flash memory or other solid-state memory technology, compact disc ROM (“CD-ROM”), or other optical storage, magnetic cassettes, magnetic tape, magnetic disk storage or other magnetic storage devices, or any other medium that can be used to store the desired information in a non-transitory fashion.,

The computing device 800 may operate in a networked environment using logical connections to remote computing devices and computer systems through a network, such as the local area network. The computing device 800 may include functionality for providing network connectivity through a NIC, such as a gigabit Ethernet adapter. The NIC is capable of connecting the computer 800 to other computing devices over the network. It should be appreciated that multiple NICs may be present in the computer 800, connecting the computer to other types of networks and remote computer systems.

The computing device 800 may also include other output (I/O) components. The output components 810 can include a visual display, an audio display, or any combination of these. Examples of a visual display can include a liquid crystal display (LCD), a light-emitting diode (LED) display, and a touch-screen display. An example of an audio display can include an audible indicator.

The non-transitory computer-readable storage medium may store processor executable instructions that, when executed by the processor, cause the processor to perform operations including determining at least one property of the device and controlling a function of the device based on the at least one property. For example, the at least one property can include an orientation of the device, a battery life, an indication of the sterilization procedure, a status of a light output from the light emitting diode, a proximity or connection to a charging device or circuit, or any combinations thereof. In some embodiments, the functions of the device to be controlled by the processor can include activating emission from the light emitting diode, stopping emission from the light emitting diode, disabling or blocking emission from the light emitting diode, activating charging of an energy storing device, stopping charging of an energy storage device, or activating an audible alarm or other indicator. For example, the processor may execute instructions relating to a sterilization procedure. The sterilization procedure can include, for example, a procedure for generating emission using the LED followed by a period of no-emission. Examples of various sterilization procedures are described in more detail below.

In some embodiments, the device may receive instructions from a mobile application executing on a mobile or hand-held device or other network-connected or wireless device. For example, the mobile device can include a phone, a hand-held computer, tablet, or other device that is mobile. In some embodiments, the device may receive instructions from a second computing device, for example, from a computer station within a hospital running a program for initiating, monitoring, or evaluating the conditions of the devices throughout the hospital sinks. The instructions received from the mobile device or computer device includes a parameter associated with a sterilization protocol. In examples, the parameter comprises any of a length of time of sterilization, a frequency of sterilization, or a combination of a length of time and frequency. Instructions relating to sterilization procedures may be described in further detail below.

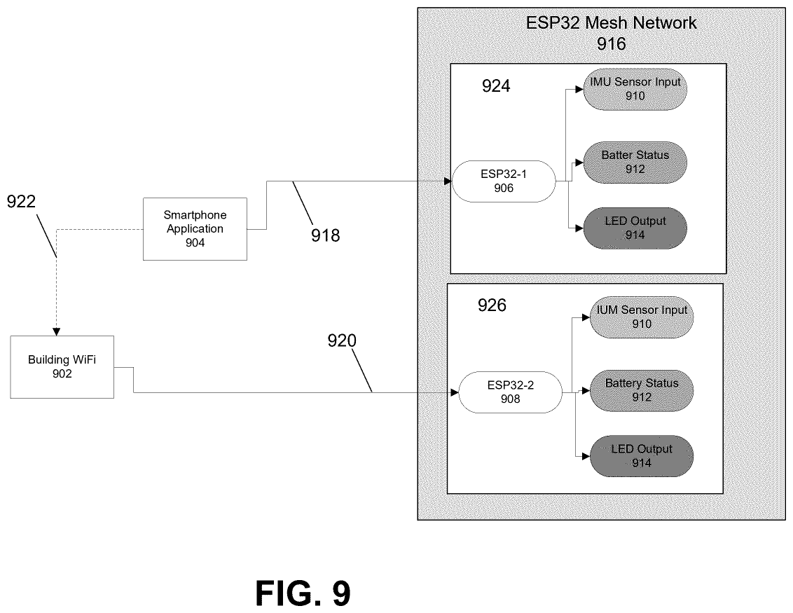

FIG. 9 provides a general schematic of a block diagram of an example network usable for implementing some aspects of the present disclosure. The block diagram may provide an example environment for operating the sink drain cover according to the embodiments described herein. The sink drain cover may be operated by use of a smartphone application 904 or by the building Wi-Fi 902. The smartphone may be connected, via an internet connection 922 to the building Wi-Fi 902. The smartphone application 904 may, via a Bluetooth connection or Bluetooth low energy (BLE) 918 connection, send instructions and or operations to the sensor and control unit 924 through ESP32-1 906. In some embodiments, the sensor and control unit 924 may be the same as the sensor and control unit 926 within ESP32-2 908 of which are within the mesh network 916. The building Wi-Fi may utilize the computing device 800 described above, to send signals to the sink drain cover over Wi-Fi connection 920. The mesh network 916 may include Imu sensor inputs 910, battery status 912 and LED output 914. For example, the IMU sensor input 910 may include an inertial sensor, an orientation sensor, or a position sensor. For example, the device may comprise an inertial sensor that may cut off or be configured to cut off power to the LED when the device is positioned upside down. The inertial device may be an added safety feature within the device to prevent undesirable UV exposure. An inertial sensor may relay information to the electronic circuit relating to a movement of the device. In some embodiments, the device may comprise an orientation sensor or a position sensor as an alternate to an inertial device. In some embodiments, the device may include all three sensors within the device for relaying a movement of the device, an orientation of the device, and a position of the device.

### Methods of Sterilization

Provided herein are methods of performing sterilization of sink drains using devices described herein. In embodiments, the method may include positioning a device discussed herein over the sink drain such that the light emitting diode is positioned for directing light into a tailpiece and/or a p-trap of the drain and subsequently initiating a sterilization protocol of the device. In embodiments, positioning may including contacting a surface of the sink or the sink drain with a lower portion of the device. The sterilization protocol may include one or more of a time duration associated with emission of light from the light emitting diode or a repetition frequency associated with emission of light from the light emitting diode.

In some embodiments, the duration of emission of light may be from 1 second to 30 minutes in duration. For example, the duration of emission of light may be at least 1 second, at least 10 seconds, at least 20 seconds, at least 30 seconds, at least 40 seconds, at least 50 seconds, at least 1 minute, at least 5 minutes at least 10 minutes, at least 15 minutes, at least 20 minutes, at least 30 minutes or 30 minutes. The sterilization time may be a function of the optical power output of the LED and the distance of the LED to the surface. For example, an LED that emits light at a wavelength range of from 260 to 270 nm, having a radiant intensity of 0.199 mW/cm², and that is positioned at a distance of about 7 cm from the surface to be sterilized, may utilize exposure times ranging from about 45 seconds to 510 seconds, such as about 50 seconds to about 505 seconds, or such as less than or about 505 seconds, less than 450 seconds, less than 400 seconds, less than 350 seconds, less than 300 seconds, less than 250 seconds, less than 200 seconds, less than 150 seconds, less than 100 seconds, less than or about 50 seconds, or greater than 50 seconds, greater than 75 seconds, greater than 100 seconds, greater than 125 seconds, greater than 150 seconds, greater than 175 seconds, greater than 200 seconds, greater than 225 seconds, greater than 250 seconds, greater than 275 seconds, greater than 300 seconds, greater than 325 seconds, greater than 350 seconds, greater than 375 seconds, greater than 400 seconds, greater than 425 seconds, greater than 450 seconds, greater than 475 seconds, greater than 500 seconds, or any ranges or values therebetween, to obtain a Log 1 to Log 5 reduction bacterial growth. For example in embodiments, one or times according to the above may be utilized to achieve, a Log 2 reduction (99% reduction), a Log 3 reduction (99.9% reduction), a Log 4 reduction (99.99% reduction), and/or a Logs reduction (99.999%). In embodiments, a Log 2 reduction (99% reduction) may be achieved from about 45 seconds to about 150 seconds, a Log 3 reduction (99.9% reduction) may be achieved at an exposure time of about 145 seconds to about 300 seconds, a Log 4 reduction (99.99% reduction) may be achieved at an exposure time of about 300 seconds to about 500 seconds, and a Logs reduction (99.999% reduction) may be predicted at an exposure time of greater than or about 495 seconds. However, other exposure ranges are contemplated as discussed above. One skilled in the art may understand that by changing the LED to a wavelength described about, the exposure time may be adjusted to achieve the same Log reduction described herein. For example, by changing the LED to an alternate wavelength or changing the distance of the LED to the surface, a higher or low UV dosage may be achieved. In some embodiments, a UV dosage of about 100 mJ/cm² may be used to achieve a Logs reduction.

The repetition frequency associated with emission of light from the light emitting diode can include daily repetitions, such as at least 1 repetition to 30 repetitions per day. For example, the daily repetition frequency may be 1 repetition, 2 repetitions, 3 repetitions, 4 repetitions, 5 repetitions, 6 repetitions, 7 repetitions, 8 repetitions, 9 repetitions, 10 repetitions, 11 repetitions, 12 repetitions, 13 repetitions, 14 repetitions, 15 repetitions, 16 repetitions, 17 repetitions, 18 repetitions, 19 repetitions, 20 repetitions, 21 repetitions, 22 repetitions, 23 repetitions, 24 repetitions, 25 repetitions, 26 repetitions, 27 repetitions, 28 repetitions, 29 repetitions, or 30 repetitions. The repetition frequency may be on a per-time unit basis (e.g., 1 time per day, 2 times per day, 1 time per hour, 2 times per hour, etc.).

In some embodiments, the sterilization protocol corresponds to a single duration of light emission from the light emitting diode per emission period sufficient to kill, destroy, and/or render inert or non-infectious the pathogen and/or the toxins. In some embodiments, the sterilization protocol corresponds to a periodically repeating duration of light emission from the light emitting diode per day sufficient to periodically or cumulatively kill, destroy, and/or render inert or non-infectious the pathogen and/or the toxins. Alternatively, the sterilization protocol may include an on-demand duration of light emission from the light emitting diode per day for a duration of time sufficient to kill, destroy, and/or render inert or non-infectious the pathogen and/or toxin. For example, the device described above may be used one time a day for a period of time sufficient to disinfect or sterilize the drain, tailpiece, and p-trap. For example, the time duration employed during the single use function may be from 30 seconds to 30 minutes in duration. In some embodiments, when the device is employed to periodically repeat the duration of emission of light, the periodic repeats may be from 1 repetition up to 30 repetitions and the duration of each on emission cycles may be from 1 second to 30 minutes. In some embodiments, when the device is employed in a repeating function as described herein, the duration of the emission of light may not be greater than 1 minute. For example, if the repetition frequency is set to 30 cycles each lasting for a duration of 1 minute total, including 30 seconds of emission followed by 30 seconds of non-emission, the LED light may be emitting a total of 30 minutes of direct exposure on the pathogens and/or toxins thus disinfecting or sanitizing the sink drain, tailpiece, and p-trap. However, as discussed above, in embodiments, a sensor may initiate the LEDs or sequence thereof anytime a flow of liquid is detected.

The methods described herein may further include activating an indicator for communicating information about the device or electronic circuit. The indicator may be an audible indicator, a visual indicator, or a wireless signal including the information about the device or electronic circuit. For example, the information about the device corresponds to a battery charge state, an orientation of the device, an operation cycle or status, or an operation error of the device.

In some embodiments, the device may receive a signal from a mobile device or hand-held device alter the sterilization protocol via a wireless or wired communication signal. For example, a user may alter the duration of the sterilization procedure following an investigation of the sink drain and finding that a particular duration has not disinfected or sterilized the sink drain sufficient enough to provide a safe environment. In some embodiments, the sterilization procedure may be altered to provide a suitable method of sterilizing the sink drain. For example, the sterilization procedure may be altered, by a user, to switch between a single emission cycle per day, a periodically repeating sequence, or an on-demand use of the device.

In some embodiments, the device described herein may be used alone or in combination with other methods for disinfecting a sink or drain. For example, the device may be used in combination with chemicals such as bleach, acetic acid, or hydrogen peroxide, optionally in lower concentrations, amounts, or exposure durations than when the devices are not used to reduce or eliminate pathogens or toxins by UV light exposure. In some embodiments, a method for sterilizing or disinfecting a sink drain can include first treating the drain with an amount of a chemical such as hydrogen peroxide, bleach, or acetic acid and subsequently placing the device over the sink drain and allowing the device to function according to methods described further below. In some embodiments, the device described herein may be used as a single treatment method for sterilizing or disinfecting a sink or drain.

### Kits

The sink drain devices disclosed herein may be present as components of a kit (e.g., a packaged kit for commercial or other applications). In some embodiments, kits are provided for carrying out any of the methods described herein. An example kit may include a carrier container being compartmentalized to receive in close confinement one or more components of the device.

In some embodiments, the kit may include a sterilization device and a set of instructions for assembling and/or using the sterilization device. In examples, the sterilization device may include a light emitting diode generating or configured for generating a light capable of and/or configured for killing, destroying, and/or rendering inert or non-infectious a pathogen and/or a toxin, an electronic circuit configured for and/or arranged to control emission of light from the light emitting diode, and a housing for enclosing the electronic circuit and at least partially enclosing the light emitting diode, the housing shaped and sized as a drain cover and for insertion into or over a drain of a sink and allowing fluid to flow from the sink into the drain when in place in or over the drain, wherein the light emitting diode is positioned in the housing for directing the light into a tailpiece and a trap of the drain for killing, destroying, and/or rendering inert or non-infectious the pathogen and/or the toxin,

The kit may further include a packaging including compartments for storing alternative or spare bottom components that may be interchangeable to accommodate various types of sink drain configurations. In some embodiments, the kit may also include alternative or spare top components. For example, a spare top component may be included in the kit as a replacement to the original top component that may be damaged or rendered unusable due to contamination or a defect altering its integrity. In some embodiments, the kit may include a charging device that may be configured to perform wireless or wired charging of a battery of the sterilization device.

In some embodiments, the set of instructions may indicate procedures for performing wired or wireless charging of a battery included in the sterilization device. In some embodiments, the set of instruction may provide details for assembling and/or using the sterilization device. For example, the instructions may include, describe, or illustrate steps of putting together the sterilization device as described above. Additionally, the instructions may include, describe, or illustrate steps for operating the sterilization device, including but not limited to providing details on setting the duration of and frequency of light emission from the LED. In some embodiments, the instructions may include information about common information regarding common types of pathogens and/or toxins found within sink drains, tailpieces, and traps. For example, the instructions may include a name of a pathogen or toxin and common time durations that may be needed for disinfecting or sterilizing that pathogen from the sink drain to provide a user a reference point for use of the sterilization device. The instructions may include directions intended for use for replacing the bottom component to adequately fit the sterilization device to different sink drain configurations.

The kit may further include a UV test card for testing the sterilization device. In some embodiments, the UV test card may indicate the status of the light emitting diode. For example, the UV light may be emitted onto the UV test card may provide insights on the overall status of the UV light. The kit may include instructions describing or illustrating testing the UV light on the UV test card. In some embodiments, the instructions may provide a scale or some indication of the status of the UV light based upon the readout on the UV test card.

In some embodiments, the instructions included within the kit may include instructions describing operation of an audible low battery alarm of the sterilization device, operation of an inertial sensor or orientation sensor of the sterilization device, charging procedures for a battery of the sterilization device, a default sterilization protocol of the sterilization device, one or more alternative sterilization protocols of the sterilization device, or instructions for changing sterilization protocols of the sterilization device.

### Illustrative Aspects

As used below, any reference to a series of aspects is to be understood as a reference to each of those examples disjunctively (e.g., “Aspects 1-4” is to be understood as “Aspects 1, 2, 3, or 4”).

As used below, any reference to a series of examples is to be understood as a reference to each of those examples disjunctively (e.g., “Examples 1-4” is to be understood as “Examples 1, 2, 3, or 4”).

Aspect 1 is a drain cover device, comprising: a light emitting diode wherein the light emitting diode comprises an average radiant light intensity, a wavelength, or both an average radiant light intensity and a wavelength configured for killing, destroying, rendering inert, rendering non-infectious, or a combination thereof, a pathogen, a fungi, a toxin, or a combination thereof; an electronic circuit board, wherein the light emitting diode is formed on one or more surfaces of the electronic circuit board; and a housing comprising a top component comprising a first diameter, a top surface, and an underside, a bottom component affixed to the underside of the top component, and comprising a bottom surface defining a bottom component central aperture, and a second diameter, wherein the first diameter is greater than the second diameter, and a drain adapter affixed to the bottom component, and defining a drain adapter central aperture, wherein the top component and the bottom component define a central volume, wherein the electronic circuit board and the light emitting diode are disposed within the central volume.

Aspect 2 is the device of aspect 1, wherein the light emitting diode comprises an ultraviolet light having a wavelength from 180 nm to 410 nm.

Aspect 3 is the device of any one of aspects 1-2, wherein the average radiant intensity of the light emitting diode comprises about 0.100 mW/cm² to about 0.500 mW/cm².

Aspect 4 is the device of any one of aspects 1-3, comprising a plurality of light emitting diodes in communication with the electronic circuit and positioned within the central volume.

Aspect 5 is the device of any one of aspects 1-4, wherein the electronic circuit board comprises a top surface and a bottom surface, wherein the light emitting diode is formed on the bottom surface of the electronic circuit board, and wherein the bottom surface is disposed facing the bottom component central aperture.

Aspect 6 is the device of any one of aspects 1-5, wherein the electronic circuit comprises an energy storage device and a charging circuit.

Aspect 7 is the device of any one of aspects 1-6, wherein the charging circuit comprises an induction coil or a wired charging port.

Aspect 8 is the device of any one of aspects 1-7, wherein the electronic circuit comprises at least one of: an indicator for communicating information about the device or electronic circuit; an inertial sensor, an orientation sensor, or a position sensor; or a wireless transceiver.

Aspect 9 is the device of any one of aspects 1-8, wherein the indicator is an audible indicator, a visual indicator, or a wireless signal including the information about the device or electronic circuit.

Aspect 10 is the device of any one of aspects 1-9, wherein the information corresponds to a battery charge state, an orientation of the device, or an operation error of the device.

Aspect 11 is the device of any one of aspects 1-10, further comprising wherein: the top surface of the top component faces away from the bottom component, and wherein the underside comprises a mounting bracket disposed along the underside at a diameter less than the first diameter.

Aspect 12 is the device of any one of aspects 1-11, wherein the diameter at which the mounting bracket is located is within about 5% of the second diameter.

Aspect 13 is the device of any one of aspects 1-12, wherein the top surface of the top component comprises a convex shape.

Aspect 14 is the device of any one of aspects 1-13, wherein the drain adapter comprises a diameter that is within about 5% of the second diameter.

Aspect 15 is the device of any one of aspects 1-14, wherein the electronic circuit further comprises: a processor; and a non-transitory computer-readable storage medium in data communication with the processor, the non-transitory computer-readable storage medium storing processor executable instructions that, when executed by the processor, cause the processor to perform operations including: determining at least one property of the device, and controlling a function of the device based on the at least one property.

Aspect 16 is the device of any one of aspects 1-15, wherein the at least one property is an orientation of the device, a battery life, an indication of a sterilization procedure, a status of a light output from the light emitting diode, a proximity or connection to a charging device or circuit.

Aspect 17 is the device of any one of aspects 1-16, wherein the function of the device is activating emission from the light emitting diode, stopping emission from the light emitting diode, disabling or blocking emission from the light emitting diode, activating charging of an energy storage device, stopping charging or an energy storage device, or activating an audible alarm or other indicator.

Aspect 18 is the device of any one of aspects 1-17, wherein the operations further include: receiving instructions from a mobile application, wherein the instructions include a parameter associated with a sterilization protocol, wherein the parameter comprises any one of a length of time of sterilization, a frequency of sterilization, or a combination of a length of time and frequency.

Aspect 19 is a method of sterilizing a sink drain, comprising: positioning the device of any of aspects 1-18 over the sink drain, wherein the bottom surface central aperture, drain adapter central aperture, or a combination thereof are disposed within or above a surface of the sink or a surface of the sink drain; and initiating a sterilization protocol of the device.

Aspect 20 is the method of aspect 19, wherein the sterilization protocol includes one or more of: a time duration associated with emission of light from the light emitting diode; or a repetition frequency associated with emission of light from the light emitting diode.

Aspect 21 is the method of any one of aspects 19-20, wherein the sterilization protocol corresponds to: a single duration of light emission from the light emitting diode per day sufficient to kill, destroy, and/or render inert or non-infectious the pathogen and/or the toxin; a periodically repeating duration of light emission from the light emitting diode per day sufficient to periodically or cumulatively kill, destroy, and/or render inert or non-infectious the pathogen and/or the toxin; or an on-demand duration of light emission from the light emitting diode per day for a duration of time sufficient to kill, destroy, and/or render inert or non-infectious the pathogen and/or the toxin.

Aspect 22 is the method of any one of aspects 19-21, further comprising: activating an indicator for communicating information about the device or electronic circuit.

Aspect 23 is the method of any one of aspects 19-22, wherein the indicator is an audible indicator, a visual indicator, or a wireless signal including the information about the device or electronic circuit.

Aspect 24 is the method of any one of aspects 19-23, wherein the information corresponds to a battery charge state, an orientation of the device, or an operation error of the device.

Aspect 25 is the method of any one of aspects 19-24, further comprising: receiving a change to the sterilization protocol via a wireless or wired communication signal.

Aspect 26 is a kit, comprising: a sterilization device, the sterilizing device comprising: a light emitting diode wherein the light emitting diode comprises a light intensity, wavelength, or both an intensity and a wavelength configured for killing, destroying, rendering inert, rendering non-infectious, or a combination thereof, a pathogen, a fungi, a toxin, or a combination thereof; an electronic circuit board, wherein the light emitting diode is formed on one or more surfaces of the electronic circuit board; and a housing comprising a top component comprising a first diameter, a top surface, and an underside, a bottom component affixed to the underside of the top component, and comprising a bottom surface defining a central aperture, and a second diameter, wherein the first diameter is greater than the second diameter, and a drain adapter affixed to the bottom component, and defining a central aperture, wherein the top component and the bottom component define a central volume, wherein the electronic circuit and the light emitting diode are disposed within the central volume; and a set of instructions for assembling and/or using the sterilization device.

Aspect 27 is the kit of aspect 26, further comprising a charging device configured to perform wired or wireless charging of a battery of the sterilization device.

Aspect 28 is the kit of any one of aspects 26-27, wherein the set of instructions comprise one or more instructions for performing wired or wireless charging of the battery of the sterilization device using the charging device.

Aspect 29 is the kit of any one of aspects 26-28, further comprising a UV test card, wherein the instructions indicate procedures for testing the sterilization device using the UV test card.

Aspect 30 is the kit of any one of aspects 26-29, further comprising at least a second bottom component or a second drain adapter, wherein the second bottom component and/or second drain adapter comprises a second leg arrangement or second support arrangement, and wherein the instructions comprise one or more instructions for replacing the bottom component with the second bottom component, replacing the drain component with the second drain component, or a combination thereof.

Aspect 31 is the kit of any one of aspects 26-30, wherein the instructions describe operation of an audible low battery alarm of the sterilization device, operation of an inertial sensor or orientation sensor of the sterilization device, charging procedures for a battery of the sterilization device, a default sterilization protocol of the sterilization device, one or more alternative sterilization protocols of the sterilization device, instructions for changing sterilization protocols of the sterilization device, or a combination thereof.

Aspect 32 is the kit of any of aspects 26-31, wherein the sterilization device comprises the device of any of Aspect(s)s 1-18.

Aspect 33 is the kit of any of aspects 26-32, wherein the sterilization device performs or is configured to perform the method of any of Aspect(s)s 19-25

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Although specific embodiments have been described, various modifications, alterations, alternative constructions, and equivalents are also encompassed within the scope of the disclosure. Embodiments are not restricted to operation within certain specific data processing environments but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or modules are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques, including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

What is claimed is:

1. A drain cover device, comprising:
a light emitting diode wherein the light emitting diode comprises an average radiant light intensity, a wavelength, or both an average radiant light intensity and a wavelength configured for killing, destroying, rendering inert, rendering non-infectious, or a combination thereof, a pathogen, a fungi, a toxin, or a combination thereof;
an electronic circuit board, wherein the light emitting diode is formed on one or more surfaces of the electronic circuit board; and
a housing comprising
a top component comprising a first diameter, a top surface, and an underside,
a bottom component affixed to the underside of the top component, and comprising a bottom surface defining a bottom component central aperture, and a second diameter, wherein the first diameter is greater than the second diameter, and
a drain adapter affixed to the bottom component, and defining a drain adapter central aperture,
wherein the top component and the bottom component define a central volume, wherein the electronic circuit board and the light emitting diode are disposed within the central volume.

2. The device of claim 1, wherein the light emitting diode comprises an ultraviolet light having a wavelength from 180 nm to 410 nm.

3. The device of claim 1, wherein the average radiant intensity of the light emitting diode comprises about 0.100 mW/cm² to about 0.500 mW/cm².

4. The device of claim 1, comprising a plurality of light emitting diodes in communication with the electronic circuit and positioned within the central volume.

5. The device of claim 1, wherein the electronic circuit board comprises a top surface and a bottom surface, wherein the light emitting diode is formed on the bottom surface of the electronic circuit board, and wherein the bottom surface is disposed facing the bottom component central aperture.

6. The device of claim 1, wherein the electronic circuit comprises an energy storage device and a charging circuit.

7. The device of claim 6, wherein the charging circuit comprises an induction coil or a wired charging port.

8. The device of claim 1, wherein the electronic circuit comprises at least one of:
an indicator for communicating information about the device or electronic circuit;
an inertial sensor, an orientation sensor, or a position sensor; or
a wireless transceiver.

9. The device of claim 8, wherein the indicator is an audible indicator, a visual indicator, or a wireless signal including the information about the device or electronic circuit.

10. The device of claim 8, wherein the information corresponds to a battery charge state, an orientation of the device, or an operation error of the device.

11. The device of claim 1, further comprising wherein:
the top surface of the top component faces away from the bottom component, and wherein the underside comprises a mounting bracket disposed along the underside at a diameter less than the first diameter.

12. The device of claim 11, wherein the diameter at which the mounting bracket is located is within about 5% of the second diameter.

13. The device of claim 11, wherein the top surface of the top component comprises a convex shape.

14. The device of claim 11, wherein the drain adapter comprises a diameter that is within about 5% of the second diameter.

15. The device of claim 1, wherein the electronic circuit further comprises:
a processor; and
a non-transitory computer-readable storage medium in data communication with the processor, the non-transitory computer-readable storage medium storing processor executable instructions that, when executed by the processor, cause the processor to perform operations including:
determining at least one property of the device, and
controlling a function of the device based on the at least one property.

16. The device of claim 15, wherein the at least one property is an orientation of the device, a battery life, an indication of a sterilization procedure, a status of a light output from the light emitting diode, a proximity or connection to a charging device or circuit.

17. The device of claim 15, wherein the function of the device is activating emission from the light emitting diode, stopping emission from the light emitting diode, disabling or blocking emission from the light emitting diode, activating charging of an energy storage device, stopping charging or an energy storage device, or activating an audible alarm or other indicator.

18. The device of claim 15, wherein the operations further include:
receiving instructions from a mobile application, wherein the instructions include a parameter associated with a sterilization protocol, wherein the parameter comprises any one of a length of time of sterilization, a frequency of sterilization, or a combination of a length of time and frequency.

19. A method of sterilizing a sink drain, comprising:
positioning the device of claim 1 over the sink drain, wherein the bottom surface central aperture, drain adapter central aperture, or a combination thereof are disposed within or above a surface of the sink or a surface of the sink drain; and
initiating a sterilization protocol of the device.

20. A kit, comprising:
a sterilization device, the sterilizing device comprising:
a light emitting diode wherein the light emitting diode comprises a light intensity, wavelength, or both an intensity and a wavelength configured for killing, destroying, rendering inert, rendering non-infectious, or a combination thereof, a pathogen, a fungi, a toxin, or a combination thereof;
an electronic circuit board, wherein the light emitting diode is formed on one or more surfaces of the electronic circuit board; and
a housing comprising
a top component comprising a first diameter, a top surface, and an underside,
a bottom component affixed to the underside of the top component, and comprising a bottom surface defining a central aperture, and a second diameter, wherein the first diameter is greater than the second diameter, and
a drain adapter affixed to the bottom component, and defining a central aperture,
wherein the top component and the bottom component define a central volume, wherein the electronic circuit and the light emitting diode are disposed within the central volume; and
a set of instructions for assembling and/or using the sterilization device.
